(19) United States
(12) Patent Application Publication
(10) Pub. No.: US 2020/0062738 A1
(43) Pub. Date: Feb. 27, 2020

(54) ANTI-CANCER STEMNESS DRUGS

(71) Applicants: Development Center for Biotechnology, Taipei (TW); National Yang-Ming University, Taipei (TW)

(72) Inventors: Cheng-Wen Wu, Taipei (TW); Erh-Hsuan Jiann Lin, Taipei (TW); Chi-Ying Huang, Taipei (TW); Jia-Ming Chang, Taipei (TW); Shih-Hsien Chuang, Taipei (TW); Hui-Jan Hsu, Taipei (TW); Wei-Wei Chen, Taipei (TW)

(73) Assignees: Development Center for Biotechnology, Taipei (TW); National Yang-Ming University, Taipei (TW)

(21) Appl. No.: 16/609,639

(22) PCT Filed: Apr. 30, 2018
(86) PCT No.: PCT/US2018/030300

§ 371 Date: Oct. 30, 2019

Related U.S. Application Data
(60) Provisional application No. 62/492,284, filed on Apr. 30, 2017.

(51) Int. Cl. C07D 403/14; A61P 35/00; C07D 209/14; C07D 333/58; C07D 401/12; C07D 403/12; C07D 401/10; C07D 413/12; C07D 487/04; C07D 405/12; C07D 405/14; C07D 495/04

(52) U.S. Cl. CPC C07D 403/14; A61P 35/00; C07D 209/14; C07D 333/58; C07D 401/12; C07D 495/04; C07D 401/10; C07D 413/12; C07D 487/04; C07D 405/12; C07D 405/14; C07D 403/12

(57) ABSTRACT

A compound for inhibiting BMI-1/MCL-1 having a structure of Formula (I), wherein the various groups are as described. A pharmaceutical composition for treating cancer includes an effective amount of a compound of Formula (I).

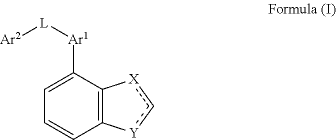

### FIELD OF THE INVENTION

This invention relates to therapeutic agents that can inhibit cancer cell sternness and the uses of these therapeutic agents in the treatment of cancers.

### BACKGROUND OF THE INVENTION

Most tumors contain heterogenous populations of tumor cells. The bulk of a tumor is made up of proliferative, differentiated cancer cells. However, some cancer cells possess properties of stem cells (i.e., sternness). These stem-cell-like cancer cells (referred to as “cancer stem cells” or CSCs) can produce new cancer cells and give rise to persistence of malignancy.

Recently, BMI-1 (B lymphoma Mo-MLV, insertion region 1 homolog) was found to be involved in cancer cell sternness. BMI-1 can regulate P16 and P19, which are cell cycle inhibitor genes. BMI-1 is elevated in several types of cancers, such as hematologic cancers and brain cancers. Reduction of BMI-1 expression levels in tumor cells can result in apoptosis and/or cell senescence and increases susceptibility to cytotoxic agents.

In addition, BMI1 is found to be rapidly recruited to sites of DNA damage. Loss of BMI1 leads to radiation sensitive and impaired repair of DNA double-strand breaks by homologous recombination.

Bmi1 is necessary for efficient self-renewing cell divisions. Bmi-1 plays a role in the maintenance of cancer stem cell populations. Kreso et al. (Nat. Med. 20, 29-36 (2014)) demonstrated that by targeting BMI1, they could eliminate human colon cancer stem cells in mouse xenografts. They further showed that a small-molecule BMI-1 inhibitor blocks tumor growth and metastasis in the absence of systemic toxicity, illustrating the feasibility of targeting self-renewal (i.e., inhibiting cancer stemness) as a new strategy for the treatment of cancers.

U.S. Patent Application Publication Nos. 2015/0315182, 2016/0214978, 2016/0280685, and 2016/0297798 by PTC Therapeutics, Inc. (South Plainfield, N.J.) disclose several inhibitors of BMI-1 and methods to treat cancers mediated by BMI-1.

Another molecule, Myeloid cell leukemia sequence 1 (MCL1) has also been found to confer resistance to chemotherapy by expanding cancer stem cells (CSCs). MCL-1 is a Bcl-2 family member and is an important anti-apoptotic protein in the development of multiple cell types.

Although prior art inhibitors of BMI-1 are known, there is a need for more effective inhibitors of BMI-1/MCL-1 for controlling cancer cell sternness.

### SUMMARY OF THE INVENTION

Embodiments of the invention relate to compounds that can inhibit BMI-1 and/or MCL-1 and can be used to treat various cancers.

One aspect of the invention relates to compounds having a structure described by formula (I) or pharmaceutically acceptable salts thereof, as inhibitors of BMI-1 and/or MCL-1 are useful in the treatment of tumor and cancer-stem-cell related diseases.

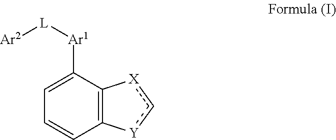

wherein

X and Y are each independently selected from the group consisting of CH₂, CH, O, S, N, and NH;

Ar¹ and Ar² are each independently selected from the group consisting of aryl and heteroaryl, wherein the aryl or heteroaryl is each optionally substituted with one or more substituents selected from R^{a} and R^{b};

L is one selected from the group consisting of: (C₁₋₆)alkyl, (C₂₋₆)alkene, CONR^{a}, NR^{a}CO, S(O)ₙNR^{a}, NR^{a}S(O)ₙ, R^{a}NCONR^{a}, R^{a}NS(O)ₙNR^{a}, R^{a}NC(S)NR^{a}, C(S)NR^{a}, NR^{a}, piperazine, O, and S;

R^{a} and R^{b} are each independently selected from the group consisting of hydrogen, halogen, (C₁₋₆)alkyl, (C₁₋₆)alkoxyl, O—(C₁₋₆)alkyl, S—(C₁₋₆)alkyl, aryl, heteroaryl, N(R^{c})(R^{d}), COR^{c}, CON(R^{c})(R^{d}), NR^{c}—CO—N(R^{c})(R^{d}), O—CO—N(R^{c})(R^{d}), NR^{c}—S(O)ₙ—N(R^{c})(R^{d}), or

R^{a} and R^{b} can join together with carbon, nitrogen or sulfur atoms, to which they are attached, to form a ring selected from the group consisting of a cycloalkyl and a heterocycloalkyl;

R^{c} and R^{d} are each independently selected from the group consisting of hydrogen, halogen, (C₁₋₆)alkyl, (C₁₋₆)alkoxyl, (C₆₋₁₉)aryl, heteroaryl, (C₃₋₁₂)cycloalkyl, or R^{c} and R^{d} can join together with carbon, nitrogen or sulfur atoms, to which they are attached, to form a 5-7 membered ring; and

n is 0, 1, or 2.

In accordance with some embodiments of the invention, a compound of the invention comprises a structure having the above described Formula I, wherein X is NH and Y is CH, or wherein X is CH and Y is NH. In any of the above embodiments, Ar¹ may be phenyl or pyridyl. In particular embodiments, X is NH and Y is CH. In any of the above embodiments, Ar¹ may be phenyl.

One aspect of the invention relates to pharmaceutical compositions for treating cancer growth, recurrence, metastasis, or resistance to therapeutics. A pharmaceutical composition in accordance with one embodiment of the invention comprises an effective amount of any one of the above described compounds having a structure depicted by Formula (I).

In accordance with embodiments of the invention, any cancer that is associated with overexpression of BMI-1 and/or MCL-1 can be prevented or treated with a compound of the invention. In accordance with some embodiments of the invention, the cancer may be a lung cancer.

Other aspects of the invention will become apparent with the following description and examples.

### BRIEF DESCRIPTION OF THE DRAWINGS

FIG. 1A shows inhibition of BMI-1 and MCL-1 expressions by lisuride. BMI1 was detected by western-blot in H1975 after treated with different concentrations of lisuride.

FIG. 1B shows inhibition of H1975 cell spheroid formation by lisuride. H1975 cells were analyzed for spheroid forming activity in serum-free matrigel, after treated with different concentrations of lisuride.

FIG. 2A shows inhibition of BMI-1 and MCL-1 expressions by compounds of the invention (lisuride derivatives). The anti-BMI1/MCL1 efficacies of Lisuride derivatives were tested in vitro by western-blot after treated in H1975 cells (10 μM, 6 h). More than 100 derivatives of Lisuride were synthesized and tested, and only a part of results was illustrated.

FIG. 2B shows that compound 44 inhibits the expressions of BMI-1 and MCL-1 in a dose-dependent manner. The anti-BMI1/MCL1 efficacy of the derivative #44 was tested by western-blot after treated in H1975 cells with different concentrations for 6 h.

FIG. 3 shows inhibition of cancer growths by various test compounds in a mouse orthotopic tumor model. Mice were orthotopically implanted with H1975-luc cells (10⁶ cells/mouse), and started to receive drug treatments for 3 weeks. The tumor growths were followed by non-invasive bioluminescent imaging, and quantified. Lisuride and Compounds #43-45 were administrated by IV injection through tail vein (1 mpk, 5 times/7 days). Tarceva (Gefitinib) was administrated orally (20 mpk, 5 times/7 days). N=5 to 7 for each group.

FIG. 4A shows a test scheme using an orthotopic mouse tumor model. Mice were orthotopically implanted with H1975-luc cells (10⁶ cells/mouse). On day 0 (defined as 2 days after tumor implantation), mice were given drug treatments for 4 weeks (5 times/week), and the tumor formations were followed by non-invasive imaging on days 14 and 28.

FIG. 4B shows imaging results of some mice in each group in the experiment described in FIG. 4A, as revealed by non-invasive imaging on days 14 and 28.

FIG. 4C shows percentages of tumor-free mice in each group on days 14 and 28.

FIG. 4D shows quantification of bioluminescent intensities of mice in each group on days 14 and 28. N=10 for Ctrl and #44 1 mpk; and n=8 for other groups.

FIG. 4E shows tumor inhibition rates in of each group on day 28.

FIG. 4F shows mouse body weights in each group, showing that there was no significant difference in mouse body weights in all groups.

FIG. 5A shows a test scheme using an orthotopic mouse tumor model. Mice were orthotopically implanted with H1975-luc cells (10⁶ cells/mouse). On day 0 (defined as 3 weeks after tumor implantation), mice were imaged and started to receive drug treatments for 3 weeks (5 times/week). The tumor growths were followed by non-invasive imaging weekly.

FIG. 5B shows imaging results of some mice in each group in the experiment described in FIG. 5A, as revealed by non-invasive imaging on days 0, 7, 14, and 21.

FIG. 5C shows relative growth rates of mice in each group. The growth rates were averaged and presented. N=7 for Ctrl and #44 1 mpk; and n=8 for other groups.

FIG. 5D shows tumor inhibition rate in of each group on day 21.

FIG. 5E shows mouse body weights in each group, showing that there was no significant difference in mouse body weights in all groups.

### DEFINITIONS

The term “alkyl” means carbon chains without double or triple bonds, and that may be linear and/or branched. An “alkyl” may be further defined by the number of carbons in the group, such as C₁-C₃ alkyl, C₁-C₆ alkyl, C₁-C₁₂ alkyl, and so on. For example, C₁-C₆ alkyl is defined as an alkyl group having 1, 2, 3, 4, 5 or 6 carbons. In this description, the number of carbons may be denoted as “C₁-C₆” or “C₁₋₆.”. Examples of alkyl groups include methyl, ethyl, propyl, isopropyl, butyl, and the like. Similarly, the term “C₀-C₄ alkyl” includes alkyls containing 4, 3, 2, 1, or no carbon atoms. An alkyl group with no carbon is a hydrogen, or a direct bond when the alkyl is a bridging moiety.

The term “alkyl” is used broadly to include “alkylenyl,” a bivalent alkyl linking two residues. Examples of bivalent “alkyl” include: —CH₂—, —CH₂—CH₂—, etc.

The term “alkene” or “alkenyl” means a linear and/or branched structure having at least one C—C double bond. An “alkene” may be further defined by the number of carbons, such as C₂-C₆ alkene, C₂-C₁₂ alkene, and so on. A C₂-C₆ alkene, for example, includes ethylene, propylene, butylenes, and the like. Similarly, “alkenyl” may be used broadly to include bivalent “alkenyl” that links two residues. A C₂-C₆ alkenyl, for example, includes ethylenyl, propylenyl, butylenyl, and the like.

The term “alkynyl” means a linear and/or branched structure having at least one C—C triple bond. An “alkynyl” group may be further defined by the number of carbons, such as C₂-C₆ alkynyl, C₂-C₁₂ alkynyl, and so on. For example, C₂-C₆ alkynyl is defined as a group having 2, 3, 4, 5 or 6 carbon in a linear and/or branched arrangement. Similarly, C₂-C₆ alkynyl includes 2-hexynyl, 2-pentynyl, or the like.

The term “alkoxy” as used herein includes an alkyl group, as defined above, connected to an oxygen atom. The term “alkoxy” also includes alkyl ether groups, where the term “alkyl” is as defined above, and “ether” means two alkyl groups with an oxygen atom between them. Examples of alkoxy groups include methoxy, ethoxy, n-propoxy, and n-butoxy.

The term “aryl,” unless specifically stated otherwise, means any stable monocyclic or fused carbon rings of up to 7 members in each ring, wherein at least one ring is aromatic. An “aryl” group may be defined by the number of carbons, such as (C₆₋₁₂)aryl, (C₆₋₁₉)aryl, and so on. Example of such aryl groups include phenyl, naphthyl, and tolyl.

The term “aryloxy” means an aryl group as defined above connected through an oxygen atom.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. An “cycloalkyl” group may be defined by the number of carbons, such as (C₃₋₆)cycloalkyl, (C₃₋₁₂)cycloalkyl, (C₃₋₁₉)cycloalkyl, and so on. Examples of cycloalkyl include cyclopropyl, cyclobutyl, cyclopentyl, cyclohexyl, adamantanyl, indanyl, indenyl, and fluorenyl.

Similarly, “cycloalkenyl” means carbocycles containing no heteroatoms and at least one nonaromatic C—C double bone. Cycloalkenyl may include mono-, bi- and tricyclic partially saturated carbocycles, as well as benzofused cycloalkenes. An “cycloalkenyl” group may be defined by the number of carbons, such as (C₃₋₆)cycloalkenyl, (C₃₋₁₂)cycloalkenyl, and (C₃₋₁₉)cycloalkenyl. Examples of cycloalkenyl include cyclohexenyl and indenyl.

The term “cycloalkyloxy” includes a cycloalkyl group as defined above connected to an oxy connecting atom.

The term “hetero,” unless specifically stated otherwise, includes one or more O, S, and/or N atoms. For example, “heterocycloalkyl” (or heterocyclyl) and “heteroaryl” include ring systems that contain one or more O, S, and/or N atoms in the ring.

The term “heterocycloalkyl” means a clycolalkyl as defined above, in which one or more ring carbons are replaced with hetero atoms, such as O, S, and/or N. Examples of heterocycloalkyl include azetidinyl, pyrrolidinyl, piperidinyl, piperazinyl, morpholinyl, and tetrahydrofuranyl. As used herein, “heterocycloalkyl” includes bridged heterocycloalkyls having two or more heterocycloalkyl groups joined via adjacent or non-adjacent atoms.

The term “heteroaryl” as used herein means a monocyclic or multicyclic ring system containing at least one aromatic ring and from one to four heteroatoms selected from N, O and/or S, wherein the nitrogen and sulfur heteroatoms may optionally be oxidized, and the nitrogen heteroatom may optionally be quaternized. Examples of a heteroaryl may include a stable 5-7 membered monocyclic- or a stable 9-10 membered fused bicyclic heterocyclic ring system, which contains an aromatic ring. The heteroaryl group may be defined by the number of carbons included therein. For example, (C₃₋₁₉)heteroaryl refers to a heteroaryl group having form 3 to 19 carbons, in addition to the hetero atom(s). Some ring(s) of a multicyclic ring system may be saturated, partially saturated, or unsaturated. A heteroaryl group includes any bicyclic or multicyclic group in which ad heterocyclic ring is fused to an aromatic ring (such as a benzene ring). The heterocyclic ring may be attached at any heteroatom or carbon atom which results in the creation of a stable structure. Examples of such heteroaryl groups include pyridine, pyrimidine, pyrazine, thiophene, oxazole, thiazole, triazole, oxadiazole, pyrrole, 1,2,4-oxadiazole, and 1,3,4-thiadiazole.

The term “heteroaryloxy” describes a heteroaryl group, as defined above, connected through an oxy connecting atom to a connecting site.

The above described ring systems, such as cycloalkyl, heterocycloalkyl, aryl, and heteroaryl, may be further connected to a non-cyclic moiety, such as an alkyl, alkenyl, or alkynyl. In these cases, the cyclic and non-cyclic parts may be separately denoted by the numbers of carbons in each part. For example, (C₃₋₁₉)heteroaryl(C₁₋₆)alkyl defines a heteroaryl ring having 3-19 carbon atoms attached to an alkyl group having 1-6 carbons. Examples of (C₃₋₁₉)heteroaryl(C₁₋₆)alkyl include, for example, furylmethyl, thienylethyl, pyrazolylmethyl, and quinoxalinylmethyl.

The term “carbamoyl” may include —NHC(O)O(C₁₋₄)alkyl and —OC(O)NH(C₁₋₄)alkyl.

The term “optionally substituted” includes both substituted and unsubstituted. For example, optionally substituted aryl could represent a pentafluorophenyl or a phenyl ring. Further, the substitution can be made at any or all subparts in a molecule. For example, a substituted aryl(C₁₋₆)alkyl may include one or more substitutions on the aryl group and/or one or more substitutions on the alkyl group.

The term “oxide” of heteroaryl or heterocycloalkyl includes, for example, N-oxides of nitrogen atoms or S-oxides of sulfur atoms. When a group is “absent,” it is “a direct bond.”

Compounds described herein having one or more double bonds may give rise to cis/trans isomers as well as other conformational isomers. The present invention includes all such possible isomers, as well as mixtures thereof.

Unless otherwise indicated by a bond symbol (dash or double dash), the connecting point to a recited group will be on the right-most stated group. That is, for example, a phenylalkyl group is connected to the main structure through the alkyl.

Compounds described herein can contain one or more asymmetric centers and may thus give rise to diastereomers and optical isomers. The present invention includes all such possible diastereomers as well as their racemic mixtures, enantiomers, all possible geometric isomers, and pharmaceutically acceptable salts thereof. The above Formula I is shown without a definitive stereochemistry at certain positions. The present invention includes all stereoisomers of Formula I and pharmaceutically acceptable salts thereof. Further, mixtures of stereoisomers as well as isolated specifics stereoisomers are also included. During the course of the synthetic procedures used to prepare such compounds, or in using racemization or epimerization procedures known to those skilled in the art, the products of such procedures can be mixtures of stereoisomers.

The compounds of the present invention are useful in various pharmaceutically acceptable salt forms. The term “pharmaceutically acceptable salts” refer to those salt forms which would be apparent to pharmaceutical chemists, e.g., those which are substantially non-toxic and which provide the desired pharmacokinetic properties, palatability, absorption, distribution, metabolism or excretion. Conveniently, pharmaceutical compositions may be prepared from the active ingredients in combination with pharmaceutically acceptable carriers.

The pharmaceutically acceptable salts may be prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper, ferric, ferrous, lithium, magnesium, manganese, potassium, sodium, zinc, and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethanmine, and the like.

When a compound of the present invention is basic, its corresponding salt can be conveniently prepared from pharmaceutically acceptable non-toxic acids, including inorganic and organic acids. Such acids include, for example, acetic, benznesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

Examples of pharmaceutically acceptable salts include mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids.

### DETAILED DESCRIPTION

Embodiments of the invention relate to compounds that can inhibit BMI-1 and/or MCL-1, which functions to promote cancer cell stemness. Compounds of the invention can be used to treat various cancers, as well as cancer recurrence and metastasis.

In search of BMI-1 and/or MCL-1 inhibitors, inventors of the present invention unexpectedly found an analog of lysergic acid, lisuride, is an effective inhibitor of BMI-1. The structure of lisuride is as shown below:

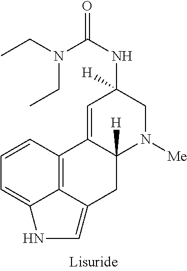

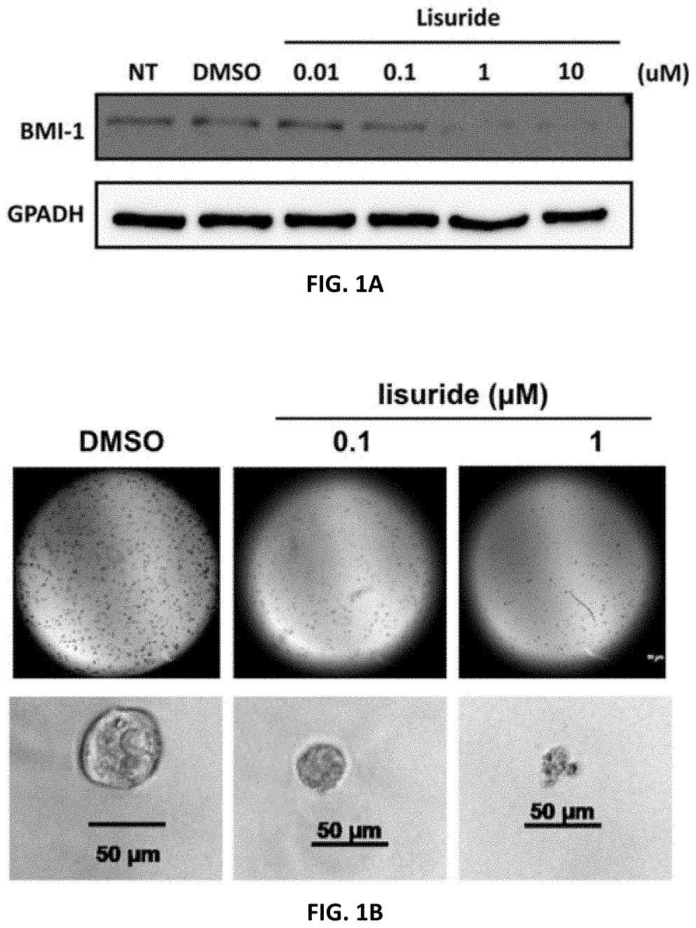

Preliminary tests confirmed that lisuride inhibited BMI-1 expression (FIG. 1A) and H1975 cell spheroid formation (FIG. 1B) in a dose-dependent manner. Lisuride is a dopamine agonist (used as an antiparkinson agent) with a structure similar to that of LSD, a lysergic acid analog. The lysergic acid analogs have a four fused-ring core structure, which may contribute to their abilities to cross the blood-brain barrier (BBB). However, for inhibition of BMI-1/MCL-1, compounds of the invention do not need to cross BBB. In fact, the ability to cross BBB may be a liability.

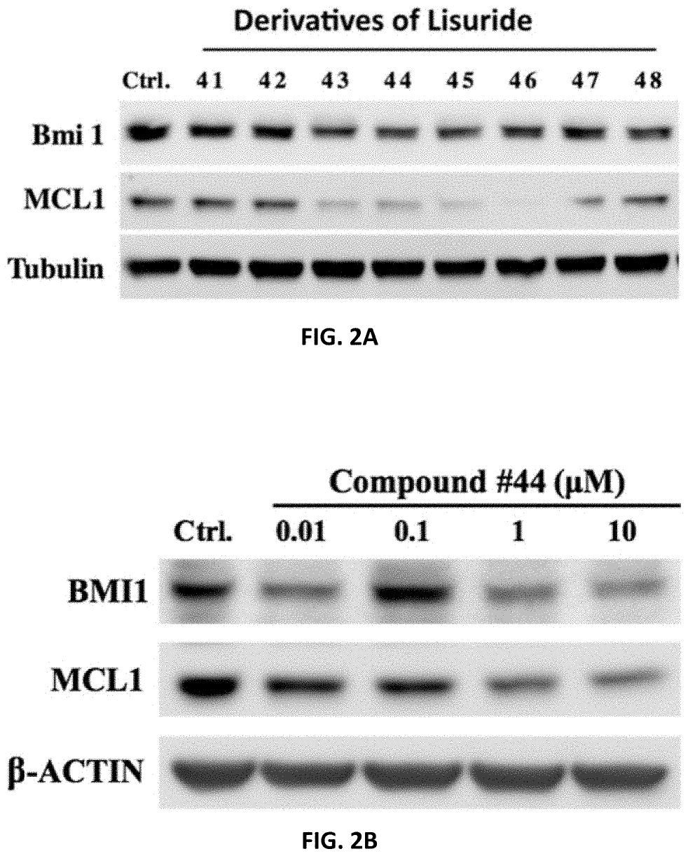

Therefore, inventors decided to improve this lead compound by breaking its four fused-ring core structure to reduce its Blood-Brain barrier (BBB) penetration ability and to improve its water-solubility. Specifically, the closed ring of the four-ring core of the lisuride was opened to reduce its planarity, and several high polarity groups were introduced to reduce its lipophilicity hydrophobicity. More than 100 derivatives of lisuride were synthesized and tested for anti-BMI-1/MCL-1 efficacy by in vitro (see FIG. 2A). The compounds of the present invention generally have an indole or other similar two fused-ring based structures (e.g., benzothiophene). These two-ring based compounds of the invention may be generalized as Formula I:

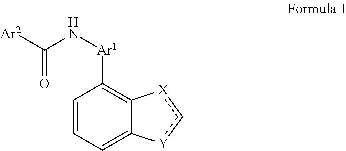

Compounds of the invention are not expected to have the ability to cross BBB. Therefore, they are less likely to have impacts on the CNS and are expected to have good BMI-1/MCL-1 inhibitory activities for use in the treatments of cancers. Compounds of the invention having a general structure of Formula I may be synthesized according to the following Scheme I:

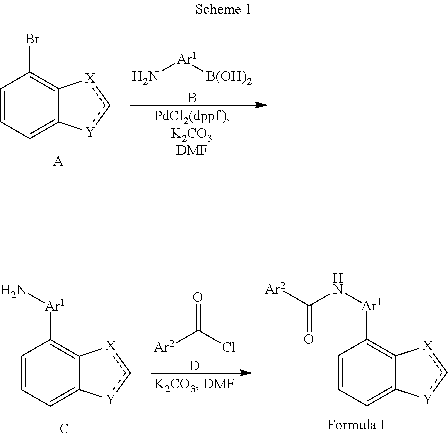

As illustrated in Scheme I, the reactions involved are conventional. First, an aromatic bromide (A) is coupled with an aryl boronic acid compound (B) under the catalysis of a palladium catalyst (e.g., PdCl₂(dppf), (1,1′-Bis(diphenylphosphino)-ferrocene)-palladium(II) dichloride, which is available commercially, for example from Sigma-Aldrich) in a reaction known as Suzuki Reaction. This coupling produces a product (C), the amino group on which can be further modified with an acyl chloride (D) to form an amide, resulting in compounds of Formula I. Because these reactions are conventional and involve commercially available reagents, one skilled in the art would be able to carry out these reactions without undue experimentation

Representative examples of compounds of Formula I are set forth below in Table 1:

TABLE 1

| Compd ID | Structure |
|---|---|
| 1 | 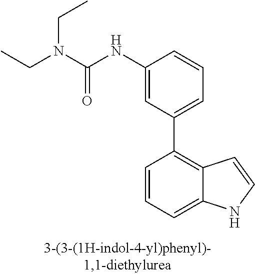 [SMILES: CCN(CC)C(=O)Nc1cccc(-c2cccc3[nH]ccc23)c1] |
| 2 | 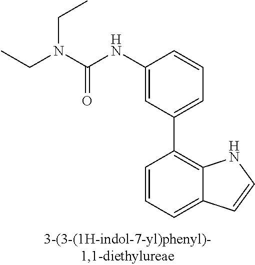 [SMILES: CCN(CC)C(=O)Nc1cccc(-c2cccc3cc[nH]c23)c1] |
| 3 | 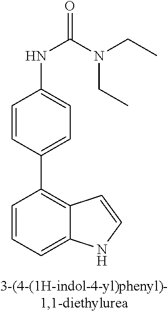 [SMILES: CCN(CC)C(=O)Nc1ccc(-c2cccc3[nH]ccc23)cc1] |
| 4 | 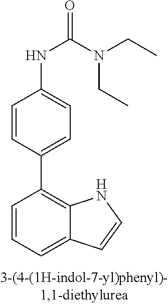 [SMILES: CCN(CC)C(=O)Nc1ccc(-c2cccc3cc[nH]c23)cc1] |
| 5 | 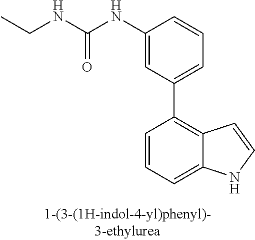 [SMILES: CCNC(=O)Nc1cccc(-c2cccc3[nH]ccc23)c1] |
| 6 | 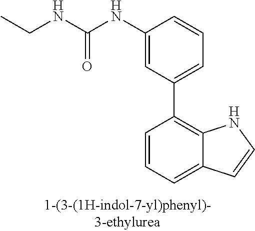 [SMILES: CCNC(=O)Nc1cccc(-c2cccc3cc[nH]c23)c1] |
| 7 | 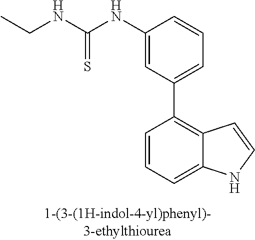 [SMILES: CCNC(=S)Nc1cccc(-c2cccc3[nH]ccc23)c1] |
| 8 | 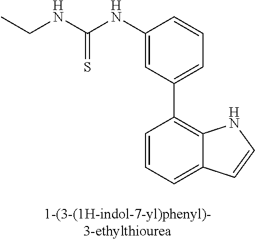 [SMILES: CCNC(=S)Nc1cccc(-c2cccc3cc[nH]c23)c1] |
| 9 | 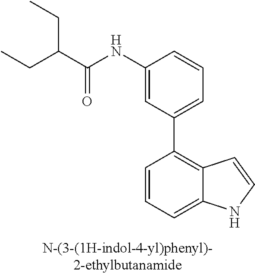 [SMILES: CCC(CC)C(=O)Nc1cccc(-c2cccc3[nH]ccc23)c1] |
| 10 | 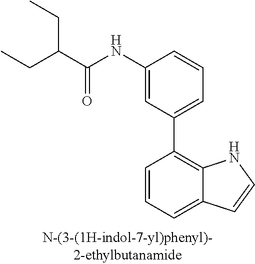 [SMILES: CCC(CC)C(=O)Nc1cccc(-c2cccc3cc[nH]c23)c1] |
| 11 | 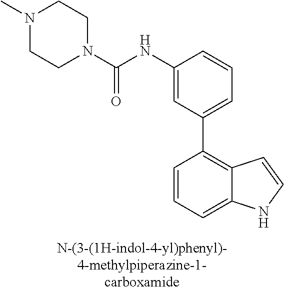 [SMILES: CN1CCN(C(=O)Nc2cccc(-c3cccc4[nH]ccc34)c2)CC1] |
| 12 | 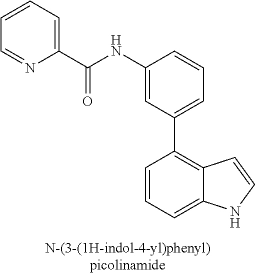 [SMILES: O=C(Nc1cccc(-c2cccc3[nH]ccc23)c1)c1ccccn1] |
| 13 | 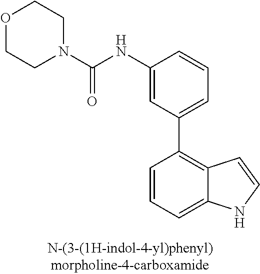 [SMILES: O=C(Nc1cccc(-c2cccc3[nH]ccc23)c1)N1CCOCC1] |
| 14 | 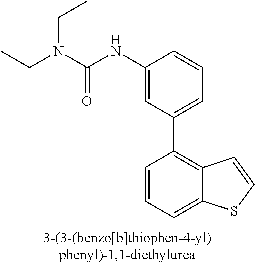 [SMILES: CCN(CC)C(=O)Nc1cccc(-c2cccc3sccc23)c1] |
| 15 | 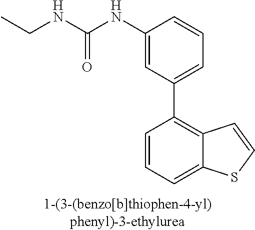 [SMILES: CCNC(=O)Nc1cccc(-c2cccc3sccc23)c1] |
| 16 | 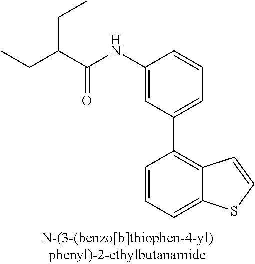 [SMILES: CCC(CC)C(=O)Nc1cccc(-c2cccc3sccc23)c1] |
| 17 | 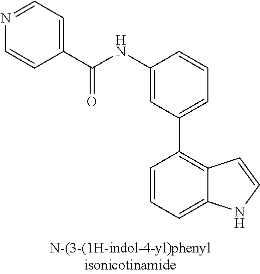 [SMILES: O=C(Nc1cccc(-c2cccc3[nH]ccc23)c1)c1ccncc1] |
| 18 | 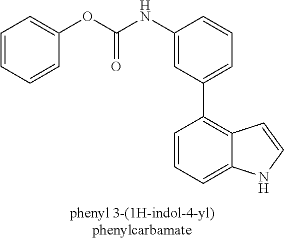 [SMILES: O=C(Nc1cccc(-c2cccc3[nH]ccc23)c1)Oc1ccccc1] |
| 19 | 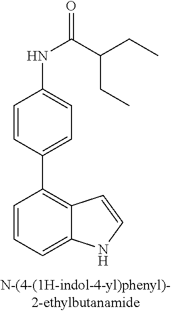 [SMILES: CCC(CC)C(=O)Nc1ccc(-c2cccc3[nH]ccc23)cc1] |
| 20 | 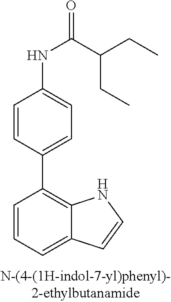 [SMILES: CCC(CC)C(=O)Nc1ccc(-c2cccc3cc[nH]c23)cc1] |
| 21 | 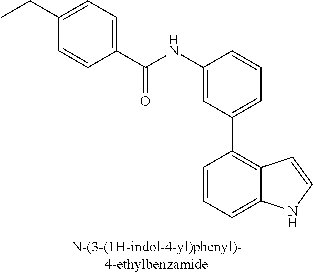 [SMILES: CCc1ccc(C(=O)Nc2cccc(-c3cccc4[nH]ccc34)c2)cc1] |
| 22 | 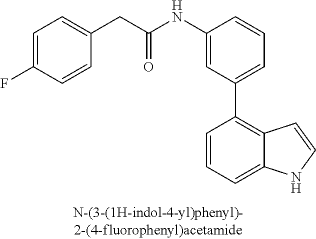 [SMILES: O=C(Cc1ccc(F)cc1)Nc1cccc(-c2cccc3[nH]ccc23)c1] |
| 23 | 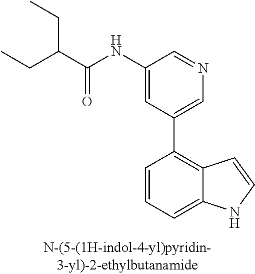 [SMILES: CCC(CC)C(=O)Nc1cncc(-c2cccc3[nH]ccc23)c1] |
| 24 | 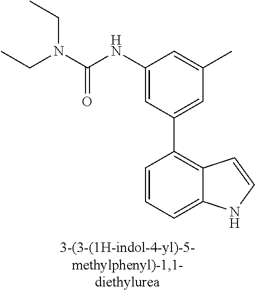 [SMILES: CCN(CC)C(=O)Nc1cc(C)cc(-c2cccc3[nH]ccc23)c1] |
| 25 | 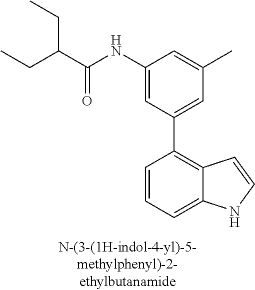 [SMILES: CCC(CC)C(=O)Nc1cc(C)cc(-c2cccc3[nH]ccc23)c1] |
| 26 | 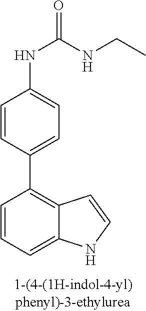 [SMILES: CCNC(=O)Nc1ccc(-c2cccc3[nH]ccc23)cc1] |
| 27 | 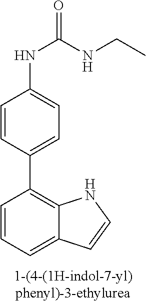 [SMILES: CCNC(=O)Nc1ccc(-c2cccc3cc[nH]c23)cc1] |
| 28 | 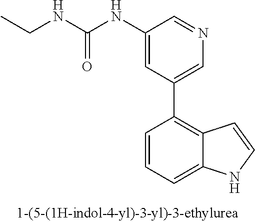 [SMILES: CCNC(=O)Nc1cncc(-c2cccc3[nH]ccc23)c1] |
| 29 | 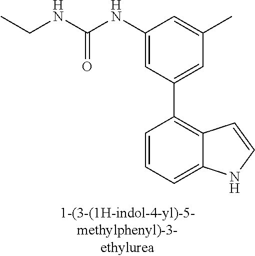 [SMILES: CCNC(=O)Nc1cc(C)cc(-c2cccc3[nH]ccc23)c1] |
| 30 | 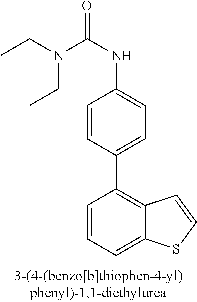 [SMILES: CCN(CC)C(=O)Nc1ccc(-c2cccc3sccc23)cc1] |
| 31 | 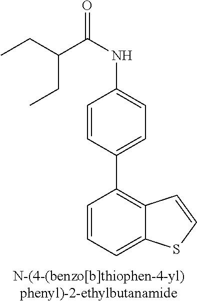 [SMILES: CCC(CC)C(=O)Nc1ccc(-c2cccc3sccc23)cc1] |
| 32 | 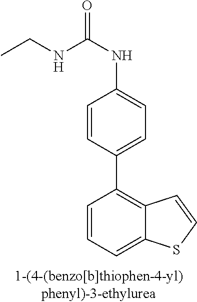 [SMILES: CCNC(=O)Nc1ccc(-c2cccc3sccc23)cc1] |
| 33 | 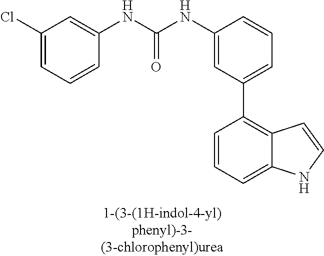 [SMILES: O=C(Nc1cccc(Cl)c1)Nc1cccc(-c2cccc3[nH]ccc23)c1] |
| 34 | 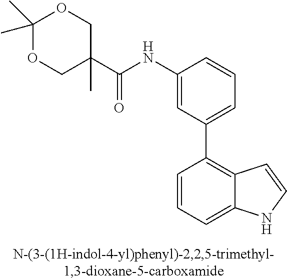 [SMILES: CC1(C)OCC(C)(C(=O)Nc2cccc(-c3cccc4[nH]ccc34)c2)CO1] |
| 35 | 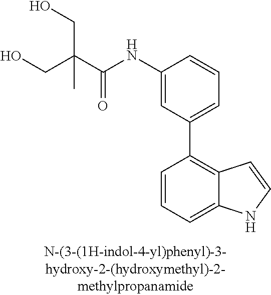 [SMILES: CC(CO)(CO)C(=O)Nc1cccc(-c2cccc3[nH]ccc23)c1] |
| 36 | 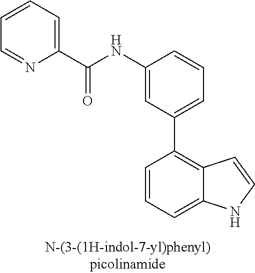 [SMILES: O=C(Nc1cccc(-c2cccc3[nH]ccc23)c1)c1ccccn1] |
| 37 | 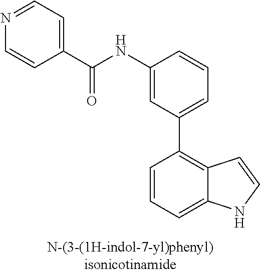 [SMILES: O=C(Nc1cccc(-c2cccc3[nH]ccc23)c1)c1ccncc1] |
| 38 | 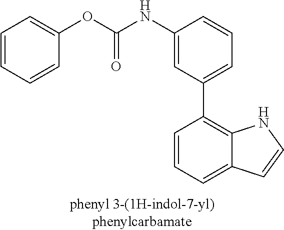 [SMILES: O=C(Nc1cccc(-c2cccc3cc[nH]c23)c1)Oc1ccccc1] |
| 39 | 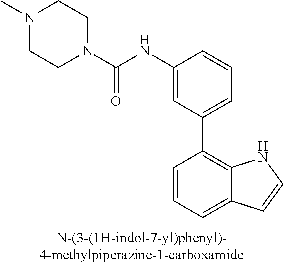 [SMILES: CN1CCN(C(=O)Nc2cccc(-c3cccc4cc[nH]c34)c2)CC1] |
| 40 | 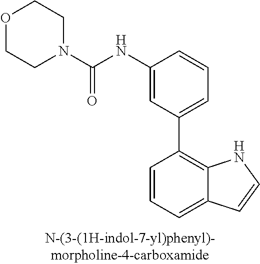 [SMILES: O=C(Nc1cccc(-c2cccc3cc[nH]c23)c1)N1CCOCC1] |
| 41 | 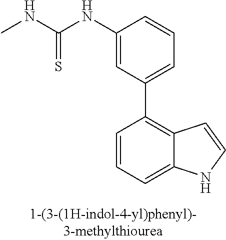 [SMILES: CNC(=S)Nc1cccc(-c2cccc3[nH]ccc23)c1] |
| 42 | 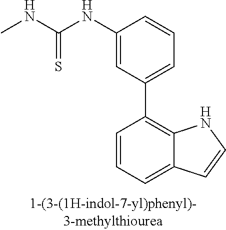 [SMILES: CNC(=S)Nc1cccc(-c2cccc3cc[nH]c23)c1] |
| 43 | 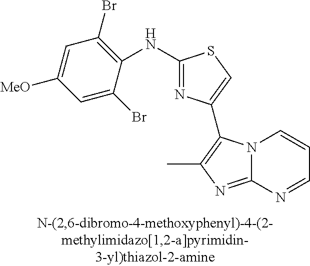 [SMILES: COc1cc(Br)c(Nc2nc(-c3c(C)nc4ncccn34)cs2)c(Br)c1] |
| 44 | 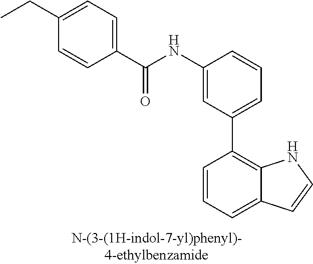 [SMILES: CCc1ccc(C(=O)Nc2cccc(-c3cccc4cc[nH]c34)c2)cc1] |
| 45 | 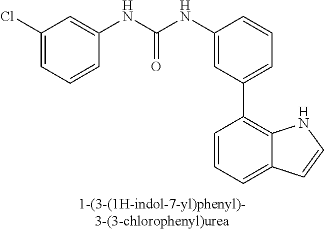 [SMILES: O=C(Nc1cccc(Cl)c1)Nc1cccc(-c2cccc3cc[nH]c23)c1] |
| 46 | 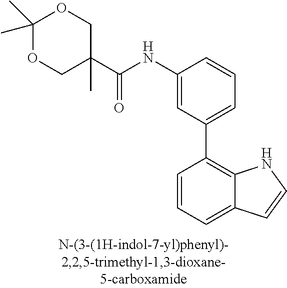 [SMILES: CC1(C)OCC(C)(C(=O)Nc2cccc(-c3cccc4cc[nH]c34)c2)CO1] |
| 47 | 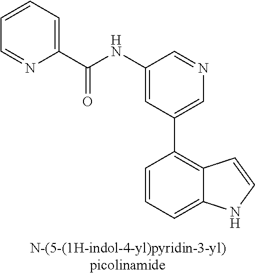 [SMILES: O=C(Nc1cncc(-c2cccc3[nH]ccc23)c1)c1ccccn1] |
| 48 | 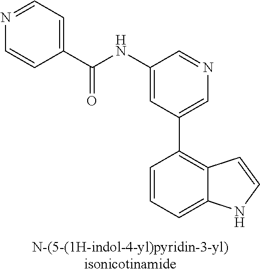 [SMILES: O=C(Nc1cncc(-c2cccc3[nH]ccc23)c1)c1ccncc1] |
| 49 | 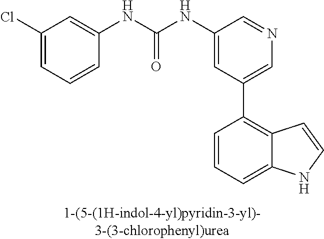 [SMILES: O=C(Nc1cccc(Cl)c1)Nc1cncc(-c2cccc3[nH]ccc23)c1] |
| 50 | 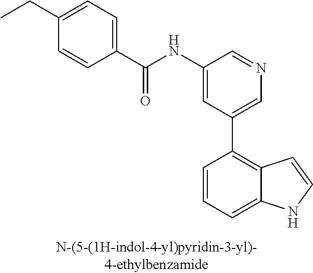 [SMILES: CCc1ccc(C(=O)Nc2cncc(-c3cccc4[nH]ccc34)c2)cc1] |
| 51 | 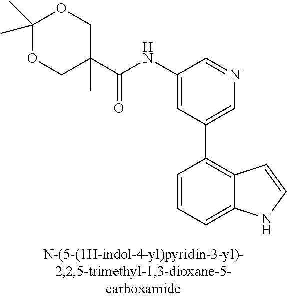 [SMILES: CC1(C)OCC(C)(C(=O)Nc2cncc(-c3cccc4[nH]ccc34)c2)CO1] |
| 52 | 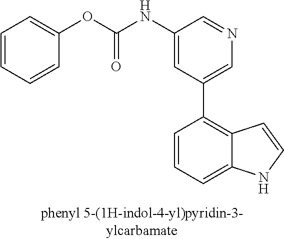 [SMILES: O=C(Nc1cncc(-c2cccc3[nH]ccc23)c1)Oc1ccccc1] |
| 53 | 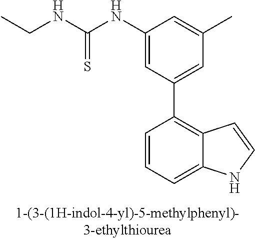 [SMILES: CCNC(=S)Nc1cc(C)cc(-c2cccc3[nH]ccc23)c1] |
| 54 | 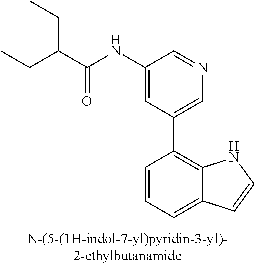 [SMILES: CCC(CC)C(=O)Nc1cncc(-c2cccc3cc[nH]c23)c1] |
| 55 | 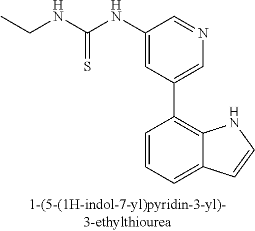 [SMILES: CCNC(=S)Nc1cncc(-c2cccc3cc[nH]c23)c1] |
| 56 | 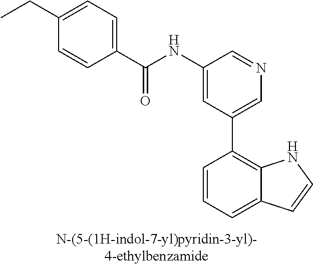 [SMILES: CCc1ccc(C(=O)Nc2cncc(-c3cccc4cc[nH]c34)c2)cc1] |
| 57 | 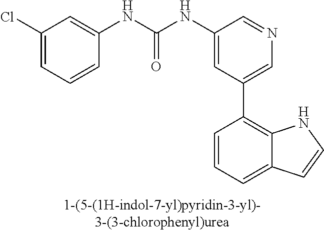 [SMILES: O=C(Nc1cccc(Cl)c1)Nc1cncc(-c2cccc3cc[nH]c23)c1] |
| 58 | 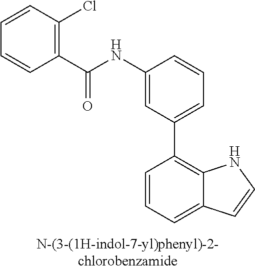 [SMILES: O=C(Nc1cccc(-c2cccc3cc[nH]c23)c1)c1ccccc1Cl] |
| 59 | 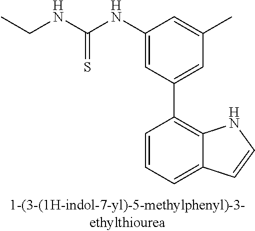 [SMILES: CCNC(=S)Nc1cc(C)cc(-c2cccc3cc[nH]c23)c1] |
| 60 | 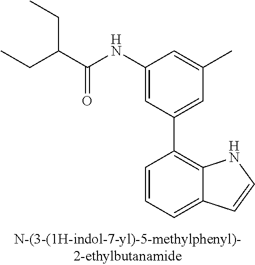 [SMILES: CCC(CC)C(=O)Nc1cc(C)cc(-c2cccc3cc[nH]c23)c1] |
| 61 | 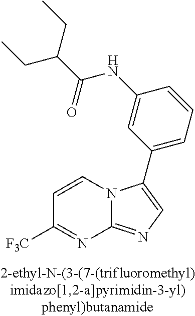 [SMILES: CCC(CC)C(=O)Nc1cccc(-c2cnc3nc(C(F)(F)F)ccn23)c1] |
| 62 | 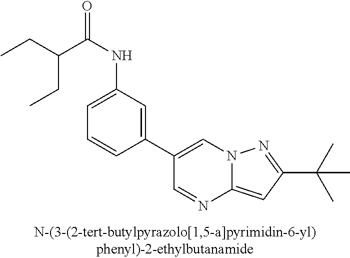 [SMILES: CCC(CC)C(=O)Nc1cccc(-c2cnc3cc(C(C)(C)C)nn3c2)c1] |
| 63 | 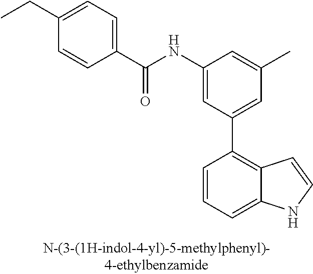 [SMILES: CCc1ccc(C(=O)Nc2cc(C)cc(-c3cccc4[nH]ccc34)c2)cc1] |
| 64 | 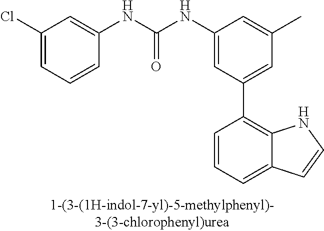 [SMILES: Cc1cc(NC(=O)Nc2cccc(Cl)c2)cc(-c2cccc3cc[nH]c23)c1] |
| 65 | 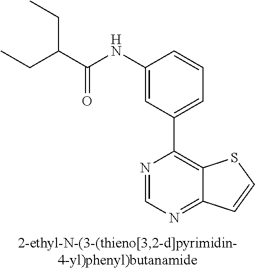 [SMILES: CCC(CC)C(=O)Nc1cccc(-c2ncnc3ccsc23)c1] |
| 66 | 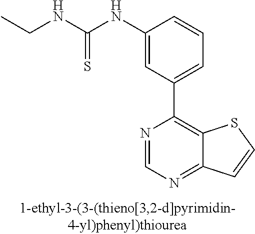 [SMILES: CCNC(=S)Nc1cccc(-c2ncnc3ccsc23)c1] |
| 67 | 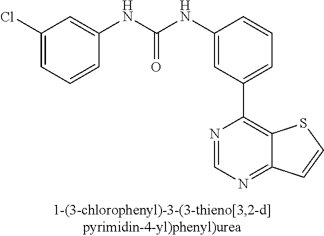 [SMILES: O=C(Nc1cccc(Cl)c1)Nc1cccc(-c2ncnc3ccsc23)c1] |
| 68 | 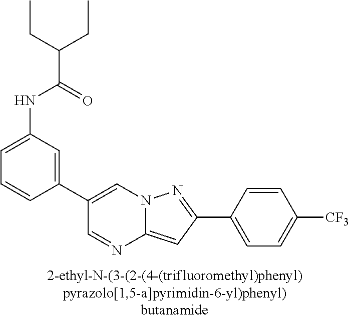 [SMILES: CCC(CC)C(=O)Nc1cccc(-c2cnc3cc(-c4ccc(C(F)(F)F)cc4)nn3c2)c1] |
| 69 | 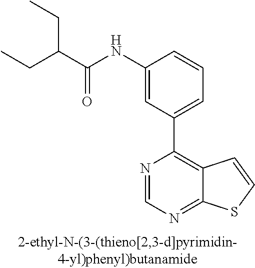 [SMILES: CCC(CC)C(=O)Nc1cccc(-c2ncnc3sccc23)c1] |
| 70 | 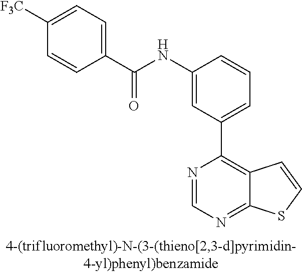 [SMILES: O=C(Nc1cccc(-c2ncnc3sccc23)c1)c1ccc(C(F)(F)F)cc1] |
| 71 | 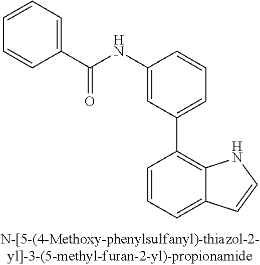 [SMILES: O=C(Nc1cccc(-c2cccc3cc[nH]c23)c1)c1ccccc1] |
| 72 | 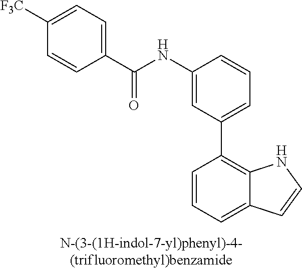 [SMILES: O=C(Nc1cccc(-c2cccc3cc[nH]c23)c1)c1ccc(C(F)(F)F)cc1] |
| 73 | 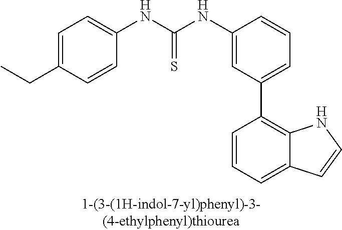 [SMILES: CCc1ccc(NC(=S)Nc2cccc(-c3cccc4cc[nH]c34)c2)cc1] |

### EXAMPLES

As noted above, the syntheses of compounds of the invention involve conventional organic reactions and use commercially available reagents. One skilled in the art would be able to synthesize these compounds without undue experimentation. The following examples are presented to illustrated certain embodiments of the present invention. These examples are for illustration only and should not be construed as limiting the scope of this invention.

Unless otherwise indicated, ¹H NMR data were obtained at 500 MHz and the compounds of this invention demonstrated efficacy in the following assays as having IC₅₀ values of less than 10 μM. The abbreviations used herein are as follows, unless specified otherwise:
Bu: butyl; Bn: benzyl; BOC: t-butyloxycarbonyl;
BOP: benzotriazol-1-yloxy tri/dimethylamino-phosphonium hexafluorophosphate;
DCC: dicyclohexylcarbodiimide; N,N-dimethylformamide;
DMAP: 4-dimethylaminopyridine;
EDC: 1-(3-dimethylaminopropyl) 3-ethylcarbodiimide hydrochloride;
EtOA:c ethyl acetate; Eq.: equivalent(s); HOBt: hydroxybenztriazole;
LAH: lithium aluminum hydride; MeOH: methanol;
MHz: megahertz; MS(ES): mass spectrophotometer-electron spray;
NMP: N-methylpyrrolidinone; Ph: phenyl;
Pr: propyl; TEA: triethylamine; THF: tetrandrofuran;
TLC: thin layer chromatography; and Tetrakis: tetrakis(triphenylphosphine)palladium.

### Example 1

### N-(3-(1H-indol-7-yl)phenyl)-4-ethylbenzamide (44)

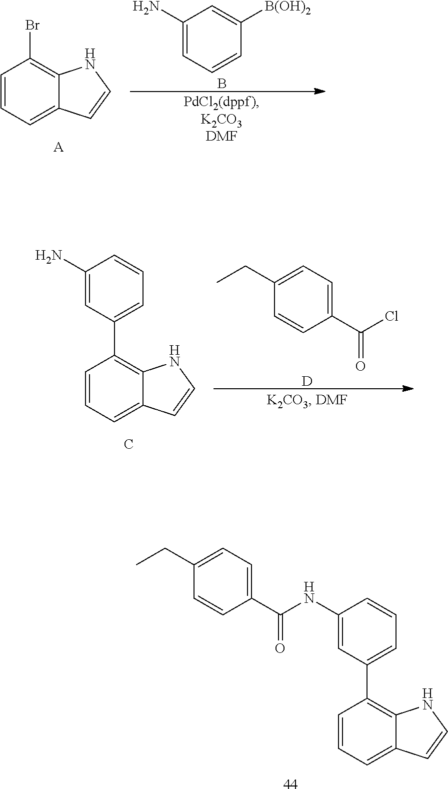

7-Bromo-1H-indole (A, 1.0 g, 5.10 mmol), 3-aminobenzeneboronic acid monohydrate (B, 948.5 mg, 6.12 mmol) and K₂CO₃ (2.82 g, 20.40 mmole) in DMF (16 ml) was degassed and then flushed with N₂. Then, PdCl₂(dppf) (416.6 mg, 0.510 mmol) was slowly added to the solution. The reaction mixture was heated to 100° C. and stirred for 5.0 hours. The reaction was monitored by TLC, and the reaction mixture was filtered with Celite. The solution was extracted twice with ethyl acetate and the organic layer was washed with brine, dried over MgSO₄(s), and concentrated under reduced pressure. The residue was purified by column chromatography on silica gel to provide 3-(1H-indol-7-yl)benzenamine (C, 964.7 mg) in 91% yield. LC/MS m/z 208.96. ¹H NMR (500 MHz, DMSO-d₆) δ 7.37-7.36 (m, 2H), 7.15-7.10 (m, 2H), 7.01 (d, J=7.2 Hz, 1H), 6.90 (s, 1H), 6.80 (d, J=7.6 Hz, 1H), 6.58-6.56 (m, 2H), and 5.12 (s, 2H).

To a solution of 3-(1H-indol-7-yl)benzenamine (C, 137.0 mg, 0.658 mmol) in DMF (2.0 ml) was added K₂CO₃ (136.4 mg, 0.987 mmol) and 4-Ethylbenzoyl chloride (0.145 ml, 0.987 mmol). The reaction mixture was stirred at 55° C. for 4.0 hours and then quenched with water. The solution was concentrated under reduced pressure, and extracted with ethyl acetate. The organic layer was washed with brine, dried over MgSO₄(s), and concentrated under reduced pressure to give N-(3-(1H-indol-7-yl)phenyl)-4-ethylbenzamide (44, 122.2 mg) as yellow solids in 55% yield. LC/MS m/z 341.60. ¹H NMR (500 MHz, DMSO-d₆) δ 10.95 (s, 1H), 10.26 (s, 1H), 8.06 (s, 1H), 7.93-7.92 (d, J=7.6 Hz, 3H), 7.57-7.56 (d, J=7.0 Hz, 1H), 7.51-7.48 (t, J=7.7 Hz, 1H), 7.39-7.36 (m, 3H), 7.3 (s, 1H), 7.13-7.11 (m, 2H), 6.54-6.53 (m, 1H), 2.72-2.67 (m, 2H), and 1.23-1.19 (m, 3H).

### Example 2

### Syntheses of Compounds 1-73 Listed in Table 1

Compounds 1-73 listed in Table 1 above were synthesized in a manner similar to that describe in Example 1. Their calculated mass and observed ESI-MS data are provided in Table 2.

TABLE 2

| Compd ID | Calculated Mass | Observed ESI-MS |
|---|---|---|
| 1 | 307.17 | 308.18 |
| 2 | 307.17 | 308.11 |
| 3 | 307.17 | 308.08 |
| 4 | 307.17 | 308.16 |
| 5 | 279.14 | 279.98 |
| 6 | 279.14 | 279.99 |
| 7 | 295.11 | 295.86 |
| 8 | 295.11 | 295.95 |
| 9 | 306.17 | 307.16 |
| 10 | 306.17 | 307.14 |
| 11 | 334.18 | 335.17 |
| 12 | 313.12 | 314.33 |
| 13 | 321.15 | 322.00 |
| 14 | 324.13 | 325.19 |
| 15 | 296.10 | 297.09 |
| 16 | 323.13 | 324.12 |
| 17 | 313.12 | 314.24 |
| 18 | 328.12 | 329.17 |
| 19 | 306.17 | 307.16 |
| 20 | 306.17 | 307.17 |
| 21 | 340.16 | 341.57 |
| 22 | 344.13 | 345.05 |
| 23 | 307.17 | 308.18 |
| 24 | 321.18 | 321.99 |
| 25 | 320.19 | 321.14 |
| 26 | 279.14 | 280.05 |
| 27 | 279.14 | 280.00 |
| 28 | 280.13 | 281.20 |
| 29 | 293.15 | 294.04 |
| 30 | 324.13 | 325.19 |
| 31 | 323.13 | 324.05 |
| 32 | 296.10 | 297.10 |
| 33 | 361.10 | 362.03 |
| 34 | 364.18 | 365.36 |
| 35 | 324.15 | 325.29 |
| 36 | 313.12 | 314.43 |
| 37 | 313.12 | 313.71 |
| 38 | 328.12 | 329.28 |
| 39 | 334.18 | 335.21 |
| 40 | 321.15 | 321.97 |
| 41 | 281.10 | 282.23 |
| 42 | 281.10 | 282.23 |
| 44 | 340.16 | 341.47 |
| 45 | 361.10 | 362.10 |
| 46 | 364.18 | 365.35 |
| 47 | 314.12 | 315.16 |
| 48 | 314.12 | 315.21 |
| 49 | 362.09 | 363.07 |
| 50 | 341.15 | 342.32 |
| 51 | 365.17 | 366.41 |
| 52 | 329.12 | 330.20 |
| 53 | 309.13 | 310.13 |
| 54 | 307.17 | 308.24 |
| 55 | 296.11 | 296.93 |
| 56 | 341.15 | 342.24 |
| 57 | 362.09 | 362.89 |
| 58 | 346.09 | 347.00 |
| 59 | 309.13 | 310.35 |
| 60 | 320.19 | 321.16 |
| 61 | 376.15 | 377.30 |
| 62 | 364.23 | 365.35 |
| 63 | 354.17 | 355.29 |
| 64 | 375.11 | 376.06 |
| 65 | 325.12 | 326.19 |
| 66 | 314.07 | 315.22 |
| 67 | 380.05 | 381.21 |
| 68 | 452.18 | 452.73 |
| 69 | 325.12 | 326.20 |
| 70 | 399.07 | 400.12 |
| 71 | 312.13 | 313.35 |
| 72 | 380.11 | 381.18 |
| 73 | 371.15 | 372.00 |

The abilities of compounds of the invention to inhibit BMI-1/MCl-1 expressions were assayed with cell cultures. BMI-1 and MCL-1 are overexpressed in many cancers, such as lung cancer cell line H1975. Therefore, cancer cells that overexpress BMI-1/MCL-1 provide a convenient platform for assaying BMI-1/MCL-1 inhibition.

In the following example, the assays use H1975 (ATCC CRL-5908), which is a human lung adenocarcinoma cell line with T790M EGFR mutation and is resistant to the first-generation Tyrosine Kinase Inhibitors, such as Gefitinib. The H1975 cells were cultured in RPMI-1640 medium, supplemented with 10% fetal bovine serum and 1% penicillin/streptomycin, in a humidified incubator at 37° C., with 5% CO₂.

### Example 1: Reduction of BMI-1 Protein Expression by Compounds of the Invention

To test inhibition of BMI-1/MCL-1 expressions, test compounds of the invention each were diluted in culture medium to reach a final concentration of 10 μM, and the cells were incubated with the medium containing these compounds for 6 hours.

After drug treatments, cells were washed twice with PBS, scraped, and resuspended in RIPA buffer containing protease inhibitors (20 mM Tris-HCl pH 7.5, 150 mM NaCl, 1 mM Na₂EDTA, 1 mM EGTA, 1% NP-40, 1% sodium deoxycholate, 2.5 mM sodium pyrophosphate, 1 mM b-glycerophosphate, 1 mM Na₃VO₄, 1 μg/ml leupeptin). Cell lysate was sonicated for 5-10 min, and centrifuged at 14000 rpm for 20 minutes at 4° C. The supernatant was collected, determined for protein concentrations, and stored at −80° C. before further use.

BMI-1/MCL-1 expression levels may be assessed with western blot analysis. For western blot analysis, all samples were diluted to an equal protein amount (50 μg), and denatured by adding 6× sample buffer (375 mM Tris-HCl, 9% SDS, 50% Glycerol, 0.03% Bromophenol blue) and heated at 95° C. for 5 minutes. The denatured protein samples were then loaded in 10% Tris-Glycine SDS polyacrylamide gel for separating proteins. The power supply for the running condition was set at 80 V for running stacking gel and 100 V for separating gel, respectively. After separation of the proteins, the protein bands were transferred from polyacrylamide gel to a nitrocellulose membrane in a transfer buffer mixture containing 10% 1× transfer stock buffer (250 mM Tris-base, 1.92M Glycine) plus 20% methanol and 70% distilled deionized water. The power supply for the transfer condition was set at 300 mA, and the transfer was carried out on ice for 2.5 hours. The nitrocellulose membranes containing the denatured proteins were blocked with a solution containing 5% skim milk at room temperature for 1 hour.

After blocking, the membranes were washed with TBS containing 0.1% Tween-20 (TBST) for 5 minutes. All membranes were incubated with the primary antibody (BMI-1 antibody, Millipore 05-637, 1:1000) at 4° C. overnight. After washing with TBST (5 min×3), the membranes were incubated with the secondary antibody (HRP-conjugated anti-mouse IgG, Jackson ImmunoReserch LABORATORIES INC., 1:5000) at room temperature for 1 hour. After washing with TBST (5 min×5), the membranes were incubated with chemiluminescence regent for 1 minute and imaged with a Bioluminiscent imaging system (Biospectrum-AC w/Bio Chemi Camera, UVP).

In addition, the membranes were also probed with antibodies against MCL-1 and tubulin. Tubulin functions as an internal control to assess the relative loadings in different lanes on the gel. Myeloid cell leukemia 1 (MCL1) is a pro-survival protein overexpressed in many cancers.

As shown in FIG. 2A, BMI-1 and MCL-1 protein expressions were significantly reduced upon treatments with compounds of the invention, particularly compounds 43, 44, and 45. These results indicate that compounds of the invention indeed are potent inhibitors of BMI-1 and MCL-1. Therefore, these compounds should be useful in the control of stemness of cancer stem cells. Accordingly, these compounds should be useful in the treatments of cancers that have been found to be associated with overexpression of BMI-1 and/or MCL-1.

FIG. 2B shows that the inhibition of BMI-1 and MCL-1 expression by compounds of the invention occurred in a dose-dependent manner. Using compound #44 (BI-44) as an example, this compound effectively inhibited the expressions of BMI-1 and MCL-1 at sub-μM concentrations.

### Example 2. In Vivo Anti-Tumor Activities of Compounds of the Invention

The derivatives showing potent anti-BMI-1/MCL-1 effects, such as compounds #43-45 (BI-43-BI-45), were selected for further in vivo anti-tumor test. The in vivo mouse tumor model was established as described below.
1. SCID mice (70 mice, about 6-6 weeks old) were quarantined for one week before lung cancer cells are implanted into their chest cavities.
2. Before implanting the cancer cells into the mouse chest cavities, the mice were anesthetized with gas anesthesia. The mice were placed in a transparent acrylic box of 20 cm×10 cm×10 cm, which was connected with a flexible tubing to an anesthesia machine and an oxygen tank. Then, an appropriate amount of anesthesia (Isoflurane) was introduced into the anesthesia machine and the valve is open. The oxygen concentration was controlled at 32-36%, and the flow rate was 0.5-1 L/min, such that the concentration of the anesthesia was within the range of 3-4%.
3. The mice were completely anesthetized after about 60-90 seconds. Then, the intra-thoracic procedures could be performed with a 29 G, 0.5 mL insulin syringe. H1975-Luc, the H1975 cells that were stably transduced with a luciferase expression vector. An aliquot of 0.1 mL cell suspension (H1975-Luc) was withdrawn and injected into the mouse chest cavity at a location on the right side between the front limb and the diaphragm. The procedure was finished within 30 seconds.
4. After the procedure, the mice were returned to the cage and they would awake in about 30-60 seconds.
5. One week after intra-thoracic implant of cancer cells, a luminescence reagent (D-luciferin) was injected into the abdominal cavities of the mice. A non-invasive in vivo imaging system (IVIS) was used to analyze bioluminescence.

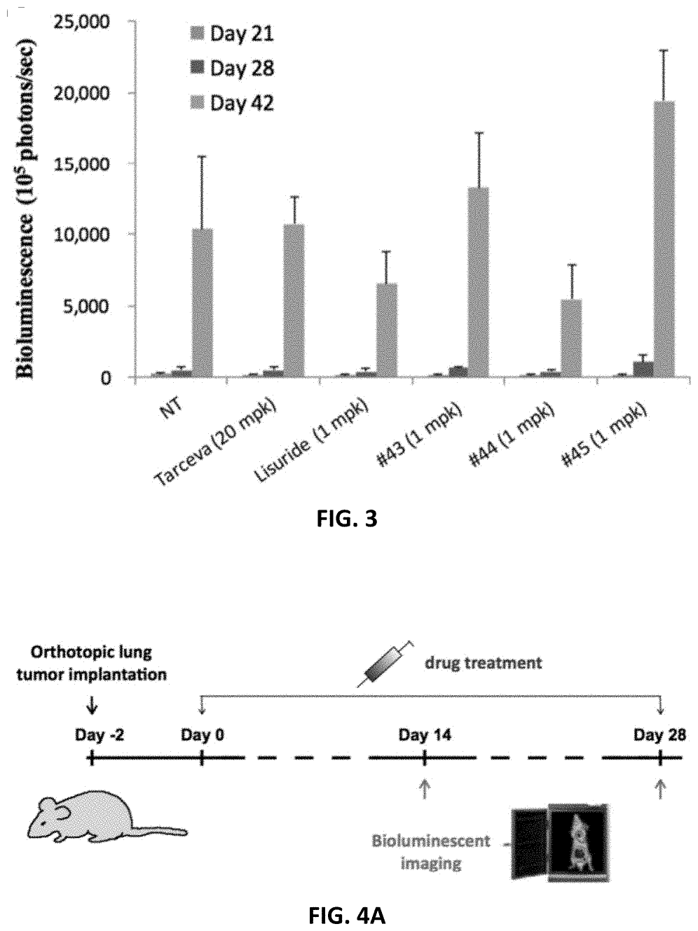

After establishment of the tumors in mice, the test compounds were administered at the indicated doses and the tumor sizes were monitored using bioluminescence. The results of tumor sizes (as measured by bioluminescence intensities) were shown in FIG. 3.

In this experiment, TARCEVA® (Erlotinib), which is a tyrosine kinase inhibitor and known to inhibit the growths of several cancer cells (e.g., non-small cell lung carcinoma, pancreatic cancer), was used (20 mg/Kg or 20 mpk) as a positive treatment control. Lisuride and BI43-45 are each used at 1 mpk. As shown in FIG. 3, among the test compounds, BI-44 showed the most significant anti-tumor growth effects.

### Example 3: Antitumor Activity of Compounds of the Invention in Orthotopic Cancer Model in Mice

Because compound 44 (BI-44) showed most potent anti-BMI-1 and MCL-1 activities, its efficacy was further examined in 2 orthotopic xenograft animal studies. The mouse cancer models were generated as described above. Orthotopic H1975-Luc model was described as previous study. BI-44 was administrated by IV injection through tail vein, 5 times/7 days, with the doses indicated on the figure. Gefitinib and Afatinib were administrated orally, 5 times/7 days, with the dose of 20 mpk (mg/Kg).

In the first animal model, Mice were orthotopically implanted with H1975-luc cells (10⁶ cells/mouse). On day 0 (defined as 2 days after tumor implantation), mice were started to receive drug treatments for 4 weeks (5 times/week), and the tumor formations were followed by non-invasive imaging on day 14 and 28.

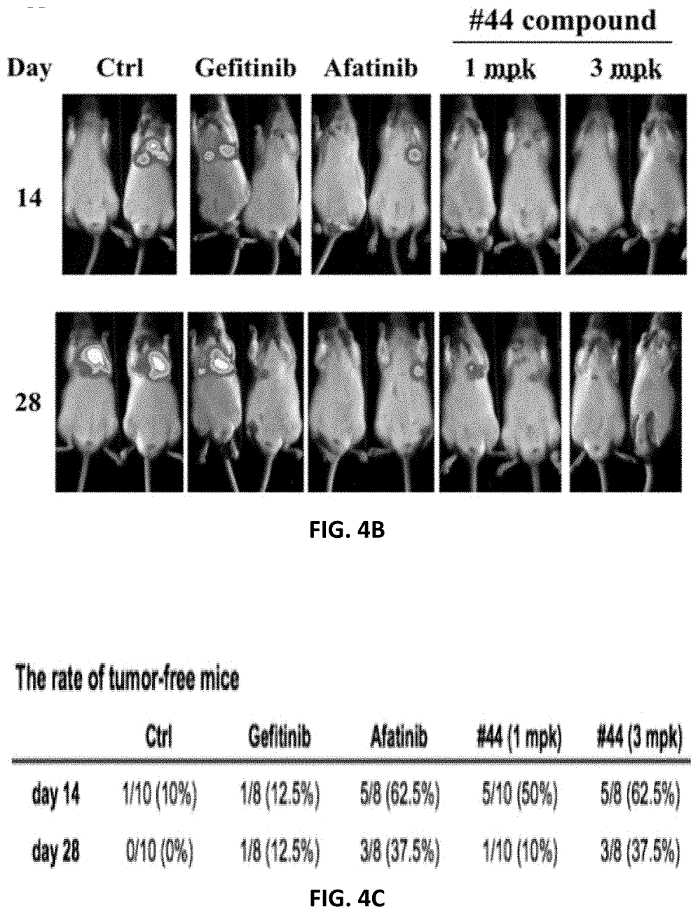

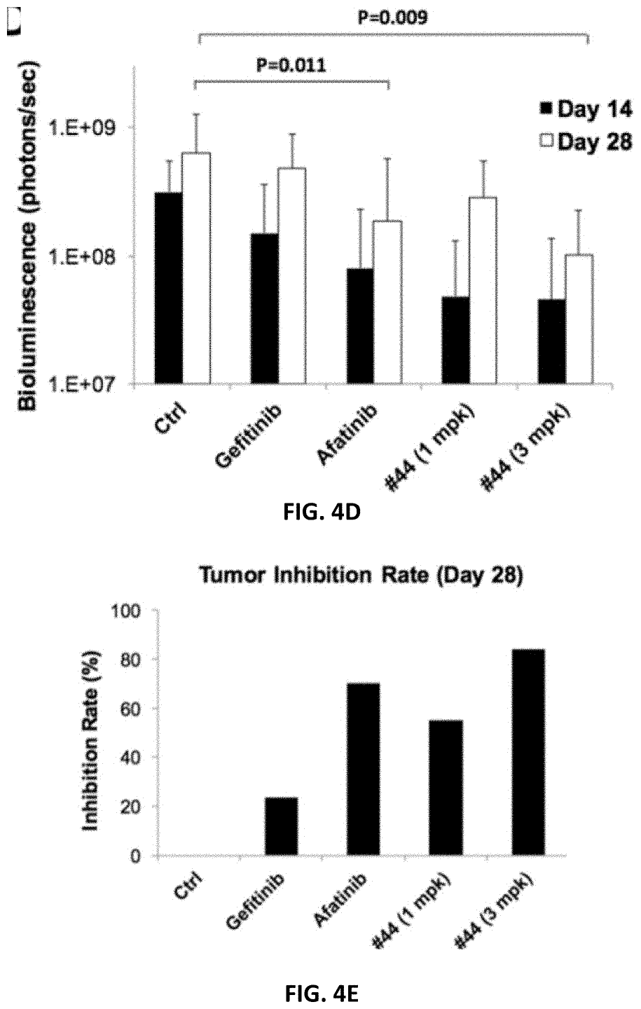

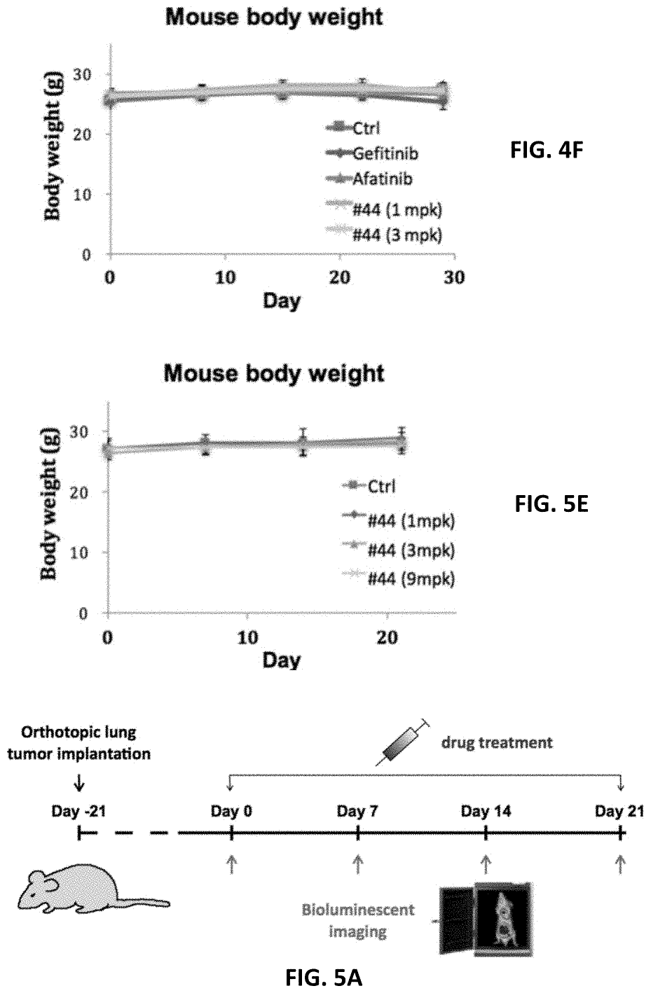

BI-44 was administrated starting 2 days after orthotopic lung tumor implantation according to the administration scheme shown in FIG. 4A, and the tumor formations were evaluated via non-invasive bioluminescent imaging on days 14 and 28.

The drugs Gefitinib (1^{st}-generation TKI, which does not target EGFR T790M) and Afatinib (2^{nd}-generation TKI, which can target EGFR T790M) were used as negative and positive controls, respectively, because H1975 contains a T790M mutation on EGFR.

A shown in FIG. 4B, which shows quantification of bioluminescent intensities of the mice in each group (N=10 for each Ctrl and BI-44 1 mpk, and n=8 for each other groups), the results showed that the group treated with BI-44 with a dose of 3 mg per kg of body weight (3 mpk) had a tumor-free rate of 62.5% (5/8) and 37.5% (3/8) on day 14 and 28, respectively, comparable to the results of the group treated with Afatinib (FIG. 4C).

Quantification of the images showed significantly reduced bioluminescent intensities in mice lungs in both BI-44 (3 mpk) and Afatinib treatment groups (FIG. 4D), with tumor inhibition rates around 80%, as compared to control (FIG. 4E). All the mice in the treatment groups did not change mice body weights during the experiments (FIG. 4F).

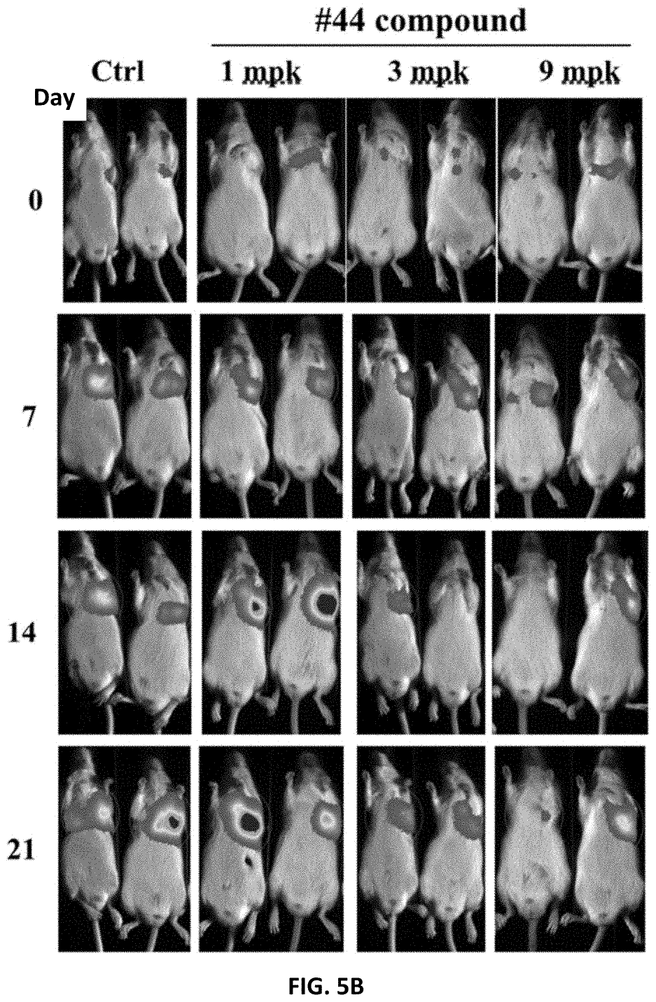

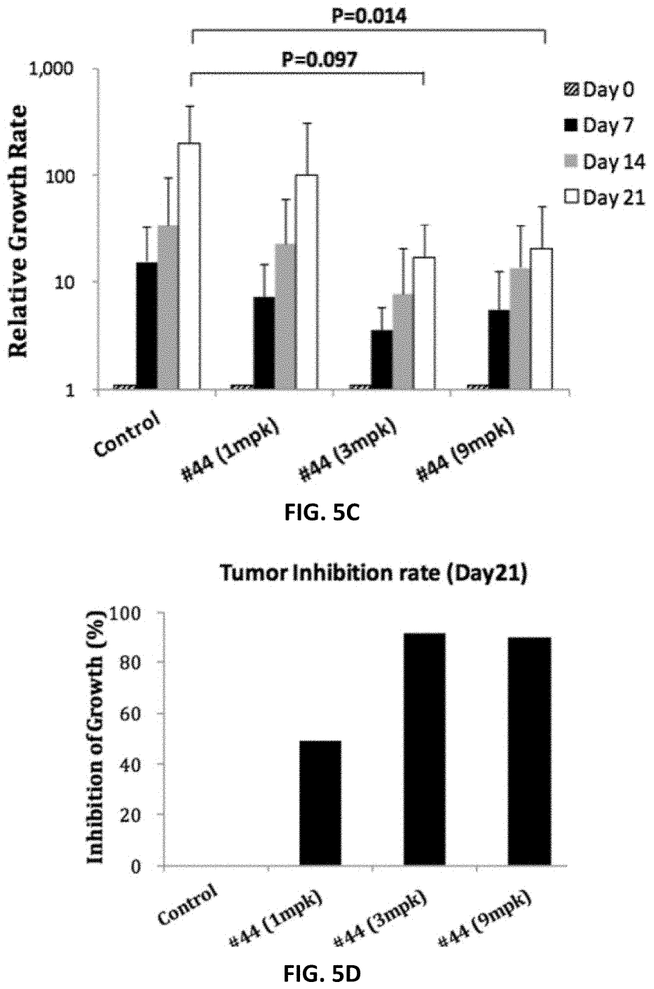

In the second model, BI-44 was administrated starting 3 weeks after tumor implantation (FIG. 5A) when all the mice contained defined luciferase signals in lungs. Then, tumor growths were followed for 3 weeks (FIG. 5A). Mice were orthotopically implanted with H1975-Luc cells (10⁶ cells/mouse). On day 0 (defined as 3 weeks after tumor implantation), mice were imaged and started to receive drug treatments for 3 weeks (5 times/week). The tumor growths were followed by non-invasive imaging weekly.

The results showed that BI-44 significantly inhibited tumor growth in a dose-dependent manner, based on imaging results of some mice in each group (FIG. 5B and FIG. 5C). The inhibition rates of 3 and 9 mpk on day 21 were around 90%. The relative growth rates of the mice in each group were averaged and presented in FIG. 5D (N=7 for each Ctrl and BI-44 1 mpk, and n=8 for each other groups). All the mice in the treatment groups did not change mice body weights during the experiments (FIG. 5E).

In sum, results from these studies show that compounds of the invention can inhibit tumor growth in vivo, presumably by inhibiting BMI-1/MCL-1 functions. These results support that compounds of the invention that can inhibit BMI-1/MCL-1 expression can indeed inhibit tumor growth. Because BMI-1/MCL-1 overexpression is associated with recurrence, metastasis, and resistance of cancers, compounds of the invention can also be used to prevent the recurrence, metastasis, or resistance of cancer cells.

For clarity of illustration, the above examples use BI-44 and lung cancer to demonstrate embodiments of the invention. However, other compounds of the invention have been shown to inhibit BMI-1 and/or MCL-1 expressions. These compounds can also be used to prevent or treat various cancers, including lung cancers and other cancers that are associated with BMI-1 and/or MCL-1 over-expressions.

Advantages of embodiments of the invention may include one or more of the following. Compounds of the invention are novel chemical entities and yet they possess BMI-1/MCL-1 inhibitory activities. Compounds of the invention have chemical structures that are different from known BMI-1 inhibitors (Nature Medicine, 20: 29-36, 2014).

Compounds of the invention can inhibit BMI-1/MCL-1 and can be used to treat cancers. In preliminary studies, compounds of the invention have superior properties to those of Afatinib, an FDA approved drug for treating non-small cell lung adenocarcinoma. Currently, treatments of non-small cell adenocarcinoma are based on tyrosine kinase inhibitors that target EGFR (e.g., Afatinib). Compounds of the invention inhibits a different target, BMI-1. Therefore, compounds of the invention may be used alone or in combination of other therapeutics to treat non-small cell adenocarcinoma. In addition to treating lung cancers, compounds of the invention can also be used to treat squamous cell carcinoma, which currently does not have any effective treatments.

While embodiments of the invention have been illustrated with a limited number of examples, one skilled in the art would appreciate that these examples are for illustration only and that other modifications and variations are possible without departing from the scope of the invention. Therefore, the scope of the protection should only be limited by the attached claims.

1. A compound for inhibiting BMI-1 and/or MCL-1 having a structure of Formula (I): 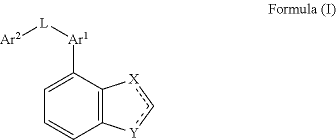
wherein
X and Y are each independently selected from the group consisting of CH₂, CH, O, S, N, and NH;
Ar¹ and Ar² are each independently selected from the group consisting of aryl and heteroaryl, wherein the aryl or heteroaryl is each optionally substituted with one or more substituents selected from R^{a} and R^{b};
L is one selected from the group consisting of: (C₁₋₆)alkyl, (C₂₋₆)alkene, CONR^{a}, NR^{a}CO, S(O)ₙNR^{a}, NR^{a}S(O)ₙ, R^{a}NCONR^{a}, R^{a}NS(O)ₙNR^{a}, R^{a}NC(S)NR^{a}, C(S)NR^{a}, NR^{a}, piperazine, O, and S;
R^{a} and R^{b} are each independently selected from the group consisting of hydrogen, halogen, (C₁₋₆)alkyl, (C₁₋₆)alkoxyl, O—(C₁₋₆)alkyl, S—(C₁₋₆)alkyl, aryl, heteroaryl, N(R^{c})(R^{d}), COR^{c}, CON(R^{c})(R^{d}), NR^{c}—CO—N(R^{c})(R^{d}), O—CO—N(R^{c})(R^{d}), NR^{c}—S(O)ₙ—N(R^{c})(R^{d}), or
R^{a} and R^{b} can join together with carbon, nitrogen or sulfur atoms, to which they are attached, to form a ring selected from the group consisting of a cycloalkyl and a heterocycloalkyl;
R^{c} and R^{d} are each independently selected from the group consisting of hydrogen, halogen, (C₁₋₆)alkyl, (C₁₋₆)alkoxyl, (C₆₋₁₉)aryl, heteroaryl, (C₃₋₁₂)cycloalkyl, or R^{c} and R^{d} can join together with carbon, nitrogen or sulfur atoms, to which they are attached, to form a 5-7 membered ring; and
n is 0, 1, or 2.

2. The compound according to claim 1, wherein X is NH and Y is CH, or wherein X is CH and Y is NH.

3. The compound according to claim 1, wherein Ar¹ is phenyl or pyridyl.

4. The compound according to claim 3, wherein X is NH and Y is CH.

5. The compound according to claim 4, wherein Ar¹ is phenyl.

6. The compound according to claim 1, wherein the compound is one selected from compounds 1-73.

7. The compound according to claim 1, wherein the compound is one selected from compounds 41-48.

8. The compound according to claim 1, wherein the compound is compound 44.

9. A method for treating a cancer, comprising: administering to a subject in need thereof a pharmaceutical composition comprising the compound according to claim 1.

10. The method according to claim 9, wherein the cancer is lung cancer.

11. The compound according to claim 2, wherein Ar¹ is phenyl or pyridyl.

12. The method for treating a cancer according to claim 9, wherein X in the compound of Formula (I) is NH and Y is CH, or wherein X is CH and Y is NH.

13. The method for treating a cancer according to claim 9, wherein Ar¹ in the compound of Formula (I) is phenyl or pyridyl.

14. The method for treating a cancer according to claim 13, wherein X in the compound of Formula (I) is NH and Y is CH.

15. The method for treating a cancer according to claim 14, wherein Ar¹ in the compound of Formula (I) is phenyl.

16. The method for treating a cancer according to claim 9, wherein the compound is one selected from compounds 1-73.

17. The method for treating a cancer according to claim 9, wherein the compound is one selected from compounds 41-48.

18. The method for treating a cancer according to claim 9, wherein the compound is compound 44.
